(19) United States
(12) Patent Application Publication
(10) Pub. No.: US 2026/0340845 A1
(43) Pub. Date: Oct. 29, 2026

(54) IMAGE MATCHING FOR SEGMENTATION AND DIAGNOSTIC PURPOSES

(71) Applicant: SYNTHESIS HEALTH INC., Vancouver, BC (CA)

(72) Inventors: Murray A. Reicher, Rancho Santa Fe, CA (US); Deepak Kaura, Calgary, Alberta (CA)

(21) Appl. No.: 19/124,220

(22) PCT Filed: Nov. 7, 2023
(86) PCT No.: PCT/US2023/079001

§ 371 Date: Apr. 24, 2025

(51) Int. Cl. G06T 7/33; A61B 6/00; A61B 6/03; A61B 6/50; G06F 40/40; G06T 7/00; G16H 10/60; G16H 15/00

(52) U.S. Cl. CPC G06T 7/337; A61B 6/03; A61B 6/481; A61B 6/501; A61B 6/5217; A61B 6/5294; G06T 7/0014; G16H 10/60; G16H 15/00; G06F 40/40; G06T 2200/24; G06T 2207/30016

(57) ABSTRACT

A system for facilitating medical image analysis can select a template to register to a patient medical image based on at least matching one or more anatomical features of the template to corresponding anatomical features of the patient medical image. The system can determine one or more patient conditions based on registration information comprising one or more numerical quantities associated with matching the one or more anatomical features.

### CROSS REFERENCE TO RELATED APPLICATIONS

Any and all applications for which a foreign or domestic priority claim is identified in the Application Data Sheet as filed with the present application are hereby incorporated by reference under 37 CFR 1.57 for all purposes and for all that they contain.

### FIELD OF THE DISCLOSURE

The present disclosure relates to the field of medical imaging and patient healthcare.

### BACKGROUND

A variety of imaging modalities exist to produce medical images of physiological tissues, anatomical structures, or body parts. Artificial intelligence algorithms can employ deep learning from hundreds or more of human-annotated medical images to segment anatomy represented on patient medical images. The process of annotating a plurality of medical images using trained professionals and training an artificial intelligence system with the annotated images to segment patient medical images is expensive, inefficient, and often difficult to adapt to a variety of circumstances.

### SUMMARY

Various implementations of systems, methods and devices within the scope of the appended claims each have several aspects, no single one of which is solely responsible for the desirable attributes described herein. Without limiting the scope of the appended claims, the description below describes some prominent features.

Details of one or more implementations of the subject matter described in this specification are set forth in the accompanying drawings and the description below. Other features, aspects, and advantages will become apparent from the description, the drawings, and the claims. Note that relative dimensions of the following figures may not be drawn to scale.

Various imaging modalities may be used to produce images of an anatomical region or body part. For example, a CT scan may produce one or more patient images of a head. Images of a particular anatomical regions of the patient images can be labelled with anatomical information that are included in a template of the anatomical region that is deformably and/or non-deformably registered to patient images. Registering a template can include applying one or more transformations to the template to match one or more anatomical features of the template to a corresponding one or more anatomical features of the patient image. As a result of registration of a template to a medical image, transformation data (which may also be referred to as “registration information” herein) indicating attributes of the transformation and/or comparisons of the registered template and the patient image, may be analyzed to determine characteristics of the medical image. For example, transformation data indicating that a particular anatomical region of the patient image is much smaller (e.g., in area or volume) than the corresponding anatomical region in the template may indicate an abnormality in that anatomical region. The transformation data may include indications of differences between anatomical features in the registered template vs. the patient image, such as in a list or table indicating differences in the area, volume, relative position (e.g., with reference to a landmark anatomical feature), absolute position (e.g. with reference to a center of the image), display characteristics (e.g., color, opacity, etc.), and/or the like. The transformation data may aid in detection and/or analysis of patient conditions, such as structural abnormalities of the patient which can be useful for generating a quantifiable diagnosis of a patient condition.

In some implementations, a template, such as borders of anatomical regions and/or anatomical information associated with those regions, can be displayed in combination with a patient image, such as overlaid on a patient image. Such displayed overlays can improve education, facilitate medial interpretation of the images, etc. A registered template can also improve medical reporting by providing anatomical labels registered to a patient medical image. Accordingly, registering a template to a patient image may be cheaper, quicker, and utilize fewer images than applying deep learning to hundreds or thousands of images yet can still provide quantifiable measures of patient conditions to improve medical diagnosing, provide improved visualization of patient medical images with anatomical information, and/or facilitate generating medical reports.

### BRIEF DESCRIPTION OF THE DRAWINGS

The following drawings and the associated descriptions are provided to illustrate implementations of the present disclosure and do not limit the scope of the claims. Aspects and many of the attendant advantages of this disclosure will become more readily appreciated as the same become better understood by reference to the following detailed description, when taken in conjunction with the accompanying drawings.

FIG. 1 is a block diagram illustrating an example computing system in communication with various devices via a network.

FIG. 2 is a flowchart illustrating an example process associated with registering templates to patient images.

FIG. 3A illustrates an example 2D image of a template with anatomical labels.

FIG. 3B illustrates an example 3D template with anatomical labels.

FIG. 4A illustrates an example patient representation.

FIG. 4B illustrates an example template that has been registered to a patient representation.

FIG. 5A illustrates an example patient image.

FIG. 5B illustrates a patient image displayed in combination with a template.

FIG. 6A illustrates an example patient image.

FIG. 6B illustrates a patient image displayed in combination with a template.

### DETAILED DESCRIPTION

Although certain implementations, embodiments, and examples arc disclosed below, the inventive subject matter extends beyond the specifically disclosed implementations to other alternative implementations and/or uses and to modifications and equivalents thereof. Thus, the scope of the claims appended hereto is not limited by any of the particular implementations described below. For example, in any method or process disclosed herein, the acts or operations of the method or process may be performed in any suitable sequence and are not necessarily limited to any particular disclosed sequence. Various operations may be described as multiple discrete operations in turn, in a manner that may be helpful in understanding certain implementations; however, the order of description should not be construed to imply that these operations are order dependent. Additionally, the structures, systems, and/or devices described herein may be embodied as integrated components or as separate components. For purposes of comparing various implementations, certain aspects and advantages of these implementations are described. Not necessarily all such aspects or advantages are achieved by any particular implementation. Thus, for example, various implementations may be carried out in a manner that achieves or optimizes one advantage or group of advantages as taught herein without necessarily achieving other aspects or advantages as may also be taught or suggested herein.

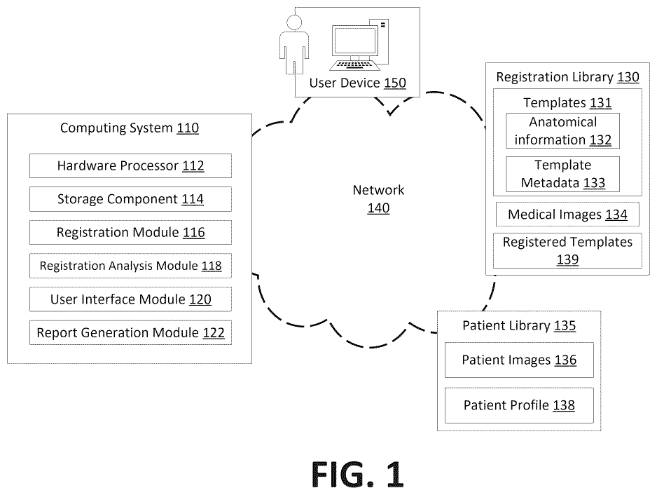

FIG. 1 is a block diagram illustrating an example computing system 110 in communication with various devices via a network 140. The network 140 can include any one or more communications networks. The network 140 can include a plurality of computers configured to communicate with one another. The network 140 can include the Internet. The network 140 may be any combination of local area network (“LAN”) and/or a wide area network (“WAN”), or the like. Accordingly, various computing devices or systems, can communicate with one another directly or indirectly via any appropriate communications links and/or networks, such as network 140 (e.g., one or more communications links, one or more computer networks, one or more wired or wireless connections, the Internet, any combination of the foregoing, and/or the like).

The registration library 130 and/or patient library 135 may comprise one or more databases. A database can be any data structure (and/or combinations of multiple data structures) for storing and/or organizing data, including, but not limited to, relational databases (e.g., Oracle databases, PostgreSQL databases, MySQL databases and the like), non-relational databases (e.g., NoSQL databases, and the like), in-memory databases, spreadsheets, as comma separated values (“CSV”) files, extensible markup language (“XML”) files, TeXT (“TXT”) files, flat files, spreadsheet files, and/or any other widely used or proprietary format for data storage. A database can include a storage device or system which can include any computer readable storage medium and/or device (or collection of data storage mediums and/or devices), including, but not limited to, one or more memory devices that store data, including without limitation, dynamic and/or static random-access memory (RAM), programmable read-only memory (PROM), erasable programmable read-only memory (EPROM), electrically erasable programmable read-only memory (EEPROM), optical disks (e.g., CD-ROM, DVD-ROM, etc.), magnetic disks (e.g., hard disks, floppy disks, etc.), memory circuits (e.g., solid state drives, random-access memory (RAM), etc.), and/or the like. Databases are typically stored in one or more data stores. Accordingly, each database referred to herein (e.g., in the description herein and/or the figures of the present application) can be understood as being stored in one or more data stores. Additionally, although the present disclosure may show or describe data as being stored in combined or separate databases, in various embodiments such data may be combined and/or separated in any appropriate way into one or more databases, one or more tables of one or more databases, and/or the like. A database may be hosted by a server. In some implementations, the registration library 130 and/or patient library 135 may include and/or be in communication with a hosted storage environment that includes a collection of physical data storage devices that may be remotely accessible and may be rapidly provisioned as needed (commonly referred to as “cloud” storage). In some implementations, one or more aspects of the registration library 130 and/or patient library 135 may be stored on the storage component 114 of the computing system 110.

The registration library 130 can comprise one or more templates 131. A template generally refers to a standardized representation or guide to delincate particular aspects of patient anatomy. A template may take various forms, such as images, lists of coordinates, or other data sets, which articulate the relative positions and sizes of anatomical features (either in 2D or 3D). These templates serve as a reference or model for identifying and understanding anatomical regions across different individuals, aiding in medical analysis, procedures, and research. Each template 131 may be associated with characteristics that allow selection of a particular template for a particular patient medical image. In some embodiments, after a template 131 is registered to a patient medical image, a registered template may be associated and/or stored with reference to the patient medical image. For example, registered templates 139 can include one or more templates to which one or more transformation has been applied to register the template to the particular patient image. As noted above, a template 131 or registered template 139 may be in various formats, such as images, lists of coordinates, or other data sets, which articulate the relative positions and sizes of anatomical features. A template 131 can include a single 2D image or a series of image slices corresponding to 2D representations of an anatomical region at parallel planes separated by a distance. A template 131 may comprise a 3D representation of an anatomical region, such as in a 3D imaging volume that may be used by a reconstruction algorithm to generate 2D slices of any plane of the 3D imaging volume. Templates 131 may have been generated according to one or more imaging modalities, such as spectroscopy, X-ray, ultrasound, MRI, CT, or the like.

The templates 131 and/or registered templates 139 may comprise and/or be associated with template metadata 133. Template metadata 133 may indicate characteristics associated with a template. Template metadata 133 can include demographic information of one or more subjects associated with the particular template. Demographics can include age, weight, height, sex, race, or the like. Template metadata 133 can include physiological conditions of one or more subjects associated with the particular template. Physiological conditions can include, for example, information associated with physiological abnormalities such as presence, size, location, and/or growth rate. Template metadata 133 can include information indicating an imaging modality of medical images that were analyzed to generate the template and/or for which the template could be registered. Template metadata 133 can indicate an anatomical region associated with a template. Templates 131 and/or registered templates 139 may be indexed and/or accessed based on their associated template metadata 133.

The registration library 130 can comprise anatomical information 132, such as information associated with one or more anatomical features represented in an image. Anatomical features can include anatomical and/or physiological structures, abnormalities, characteristics, or the like. Non-limiting examples of anatomical features of a brain region may include a skull, a brain surface, ventricles, a midline, a mass, a tumor, a growth, a hemorrhage, a blood vessel occlusion, a stroke region, a lobe, or the like. The anatomical information 132 may include name(s) of anatomical features. The anatomical information 132 may include information relating to location(s) and/or region(s) of anatomical features. The anatomical information 132 may include information relating to characteristics and/or properties of anatomical features. The anatomical information 132 may include one or more labels. The anatomical information 132 may be derived from and/or associated with the templates 131 and/or registered templates 139. In some implementations, the anatomical information 132 may be applied to the patient images 136.

The registration library 130 can include one or more medical images 134 that are associated with templates. For example, a template may be generated based on and/or include one or more medical image 134. The medical images 134 may be generated using any imaging modality and may include one or more images or videos of a subject.

The patient library 135 can comprise one or more patient images 136 and associated patient profiles 138. The patient library may be stored at various locations, such as at locations where patient imaging is performed (e.g., hospitals, imaging centers, etc.), cloud storage, and/or local to the computing system 110. The patient images and associated patient profiles 138 may be accessed by the computing system 110, e.g., transferred to the computing system 110 or a local storage device coupled to the computing system 110, for registration with one or more templates, as discussed herein. Patient images 136 may gencrated using any imaging modality and may include one or more images or videos of a subject.

A patient profile 138 may include information associated with a patient associated with one or more of the patient images 136. A patient profile 138 may include information indicating one or more characteristics of a patient. For example, a patient profile 138 may include demographic information of a patient, such as age, weight, height, sex, race, or the like. A patient profile 138 may include a medical history of a patient, such as information relating to diseases of the patient, physiological conditions of the patient, medical treatment given to the patient, medical procedures provided to the patient, medications prescribed and/or taken by the patient, or the like. Medical history can include past and/or present information, such as previous conditions of the patient and/or current conditions of the patient. A patient profile 138 may include information relating to previous templates registered to the patient's images, such as an imaging modality associated with a previous template, transformation(s) applied to previous templates to register them to the patient image, or the like. A patient profile 138 can indicate an imaging modality used to generate a patient image, such as the type of imaging modality. A patient profile 138 can indicate an anatomical region associated with a patient image.

The computing system 110 may comprise one or more computing devices. In some implementations, the computing system 110 may be in communication with and/or hosted by one or more servers, such as a server that is remote to the user device 150. In some implementations, one or more aspects of the computing system 110 may be implemented on user device 150.

The computing system 110 can include a hardware processor 112. The hardware processor 112 can include one or more processors which may be on one or more computing devices. The hardware processor 112 can be configured to execute program instructions to cause the computing system 110 to perform one or more operations. The hardware processor 112 can be configured, among other things, to process data, execute instructions to perform one or more functions, and/or control the operation of the computing system 110. The hardware can execute instructions to perform functions related to storing and/or transmitting data.

The storage component 114 can include any computer readable storage medium and/or device (or collection of data storage mediums and/or devices), including, but not limited to, one or more memory devices that store data, including without limitation, dynamic and/or static random-access memory (RAM), programmable read-only memory (PROM), erasable programmable read-only memory (EPROM), electrically erasable programmable read-only memory (EEPROM), optical disks (e.g., CD-ROM, DVD-ROM, etc.), magnetic disks (e.g., hard disks, floppy disks, etc.), memory circuits (e.g., solid state drives, random-access memory (RAM), etc.), and/or the like.

The storage component 114 can store data such as processed and/or unprocessed data, templates, patient images, registered images, anatomical information, or the like. In some implementations, the storage component 114 may store data associated with the registration library 130 and/or patient library 135. The storage component 114 can store registration information. Registration information can include data associated with registering a template to a patient image. For example, registration information can include data associated with one or more transformations applied to a template to register the template with the patient image. Registration information can include pixel data, such as one or more arrays of data and/or a 3-dimensional array of data which may comprise values indicating color, such as red-green-blue (RGB) values. Registration information can indicate differences between a registered template (e.g., a template that has been transformed to register one or more anatomical landmarks of the template to the patient image) and the patient image. In some implementations, registration is performed by transforming (e.g., resizing, rotating, etc.) a template so that anatomical landmarks of the template and patient image are aligned. As part of this registration, some anatomical regions may also be closely registered, while others may not be as closely registered, which could indicate an abnormality in the patient image. Thus, in some implementations registration information may indicate differences in area, size, color, RGB pixel values, and/or other characteristics for different anatomical segments. As one example, registration information associated with a head CT may indicate differences between the registered template and the head CT for cach of a frontal, parietal, temporal, occipital, and insular segment. In some embodiments, registration information may indicate differences between a (non-registered) template and the same template after registration with the patient image and/or between the non-registered template and the patient image.

Registration information can include an area and/or volume, such as an area of an image corresponding to pixel data having certain RGB values. Registration information can include a number or percentage of data values of pixel data (e.g., in a registered template) having certain RGB values (e.g., when compared to corresponding pixels in a patient image). For example, registration information can include a difference in a percentage and/or number of pixels between a registered template and a patient image that correspond to a same anatomical feature in the registered template and the patient image. Registration information can include a number or percentage of pixels whose data values were changed during registration.

The registration module 116 can register templates to patient images. The registration module 116 can perform deformable and/or non-deformable registration. Registering a template can include applying one or more transformations to the template, such as modifying one or more pixel values of the template to apply a rotation, tilt, translation, compression, and/or stretching of a template or various portions of a template. As noted above, registration may be applied to a particular anatomical landmark of the template and patient image, with the remainder of the template being transformed in the same manner, which may be referred to as uniform registration or a non-deformable registration. In other implementations, registration may apply a transformation non-uniformly to portions of a template, such as by separately registering individual anatomical regions of the template and patient image, which may be referred to as deformable registration.

In certain embodiments, edge-finding algorithms may be used to delineate the boundaries of anatomical regions within a patient medical image. An initial point, considered the central or expected central point of the anatomical region, may be selected as a starting reference. Multiple techniques can then be deployed to identify the boundaries of this anatomical region, including but not limited to:
Thresholding: Segmenting the image based on pixel intensity.
Region Growing: Iteratively adding neighboring pixels to a region based on predefined criteria.
Graph-based Methods: Using graph theory to segment the image into different regions.
Edge-based Methods: Detecting discontinuities in pixel intensity to identify edges.
Active Contours or Snakes: Using curves that move to minimize energy functions and identify object boundaries.
Watershed Algorithms: Transforming the image into a topographic map to isolate regions.

Once the boundaries are established, the corresponding anatomical region in the template is then deformed to closely match, or exactly match (depending on the specific embodiment), the identified area within the medical image.

In some embodiments, a data array indicting the nature and/or extent of the applied transformations, may be generated, and which in some implementations may be used to effectuate the transformation. This data array is then stored, either in association with the patient medical image or the registered template. For example, if deformable registration is performed separately for each of multiple anatomical regions in a template, the data array for the medical image may separately indicate transformations that were applied to each anatomical region. Information associated with registering templates, such as a data array, can be stored in the storage component 114.

The registration analysis module 118 can access registration information generated when registering templates. Registration information can include and/or indicate a transformation applied to a template to register the template to a patient image. The registration analysis module 118 can analyze the registration information to determine one or more patient conditions. Transformations, which may be represented as data arrays, can indicate a direction and/or magnitude associated with transforming a template, or portions thereof, to match anatomical features of the template to corresponding biological features of a patient image. For example, transformations may indicate a difference in size and/or location between various anatomical features represented in a template and a patient image. Such differences can indicate a degree to which the patient's anatomy deviates from average as represented by the template. Accordingly, the registration analysis module 118 can determine one or more patient conditions based on analyzing differences between the patient image and the template as represented by transformation(s) applied to the template to register the template to the patient image.

The user interface module 120 may generate user interface data for rendering one or more interactive graphical user interfaces. A user interface can include one or more patient images. A user interface can include one or more templates or portions of a template. A user interface can include one or more images of a template or portions of images of a template. A user interface can include anatomical information. The user interface module 120 may receive and/or process user inputs, such as user inputs received via an interactive graphical user interface. For example, a user may adjust one or more display preferences for viewing patient images, templates, and/or anatomical information.

The report generation module 122 can generate one or more reports or one or more portions of a report. For example, the report generation module 122 can generate text for a report based on anatomical information associated with a template registered to a patient image. In some embodiments, the report generation module 122 can identify a proper location in a report, such as a section of a report corresponding to an anatomical feature represented in a patient image that has been selected by a user. The report generation module 122 can access a location in a report to generate text in the location, to display to a user, and/or for a user to add text to the location of the report.

The user device 150 can comprise a computing device such as a computer, a laptop, a smartphone, a tablet, a mobile computing device, or the like. A user may interact with the computing system 110 via the user device 150. The user device 150 can comprise one or more hardware processors configured to execute program instructions to perform one or more operations. The user device 150 can comprise one or more displays configured to render one or more graphical user interfaces, such as user interfaces generated by the user interface module 120. In some implementations, the user device 150 may implement and/or execute one or more aspects of the computing system 110. In some implementations, the user device 150 may be remote to the computing system 110. In some implementations, the user device 150 may comprise one or more aspects of the registration library 130 and/or patient library 135. In some implementations, the user device 150 may be remote to the registration library 130 and/or patient library 135.

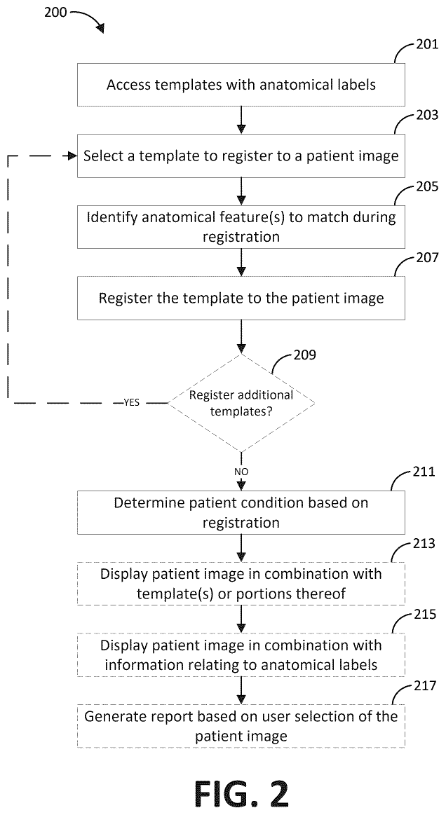

FIG. 2 is a flowchart illustrating an example process 200 associated with registering templates to patient images. This process, in full or parts, can be executed by one or more hardware processors, whether they're associated with a singular or multiple computing devices like computing system 110 and/or user device 150, and even devices in remote or wireless communication. The implementation may vary. For example, process 200 could be controlled by one or more hardware processors related to computing system 110, or can involve modifications like omitting blocks, adding blocks, and/or rearranging the order of execution of the blocks. Process 200 serves as an example and isn't intended to restrict the present disclosure.

At block 201, a computing device can access one or more templates. As noted above, a template may be in various formats, such as images, lists of coordinates, or other data sets, which articulate the relative positions and sizes of biological features. A template can comprise one or more images including series of images of an anatomical region or body part. Images of a series may correspond to 2D representations of “slices” of an anatomical region at parallel planes separated by a certain distance such as 1 mm, 2 mm, 3 mm, or the like which distance may change based on the imaging technique used and/or user preference. A series of images may be generated with a certain imaging technique. For example, a first series of images may be generated from a non-contrast CT scan and a second series of image slices may be generated from a contrast CT scan. Various imaging modalities can be used to generate images, such as computed tomography (CT), magnetic resonance imaging (MRI), spectroscopy, endoscopy, mammography, positron emission tomography (PET), X-ray, ultrasound, digital radiography, computcd radiography, and the like. Imaging modalities can also include an image type such as contrast or non-contrast.

A template can correspond to an anatomical region for each of multiple images of a series of medical images, such as slices of a 3D imaging volume. A template can include less than ten series of images, less than five series of images, less than four series of images, less than three series of images, or less than two series of images. In some implementations, a template can include a single series of images. Advantageously, generating fewer templates may reduce costs associated with generating the templates such as time, money, professional oversight, computer processing requirements, and the like.

A template can include anatomical information. Anatomical information can be associated with one or more anatomical features represented in the images of a template. Anatomical information can identify a name of an anatomical feature, a location of an anatomical feature, or the like. For example, a template comprising one or more images of a brain may include anatomical information identifying the names and locations of anatomical features or features of the brain. Anatomical information can include information associated with an anatomical feature such as physiological processes performed by the anatomical feature, common health risks associated with the anatomical feature, size of the anatomical feature, or the like. In some implementations, a computing device can generate anatomical information based on user input. For example, a medical professional can input, adjust, reassign, modify, add, delete, etc. anatomical information to the image(s) of a template. In some implementations, a computing device may automatically generate anatomical information of a template, such as based on deep learning, machine learning, neural networks, artificial intelligence, or the like.

A template may comprise one or more series of images corresponding to a single individual. A template may comprise one or more series of images corresponding to multiple individuals. For example, a template may include a first series of images of a body part of a first individual and may also comprise a second series of images of a corresponding body part of a second individual. A template may comprise one or more series of images corresponding to individual from a same demographic. For example, a template may include series of images which cach correspond to an individual of a same demographic. Demographics can include age, sex, race, ethnicity, health conditions or health history, disease, weight, height, BMI, or the like. Advantageously, a template may comprise one or more series of images representing the same or similar demographics, whether the images are from a single individual or multiple individuals.

At block 203, the computing device can select a template to register to a patient image. The computing device can select a template based on metadata associated with the templates. As noted above, template metadata can include one or more of demographic information of subjects associated with the templates, imaging modality used to generate a template, physiological conditions, or the like. The computing device can select a template based on a patient profile. As noted above, a patient profile can include one or more of patient demographic information, patient medical history, historical templates registered to the patient's image(s), imaging modality used to generate patient images, or the like. The computing device can select a template based on identifying template metadata that corresponds to a patient profile. As an example, the computing device may select a template having demographics metadata that matches the patient demographics of the patient profile. As another example, the computing device can select a template having metadata that corresponds to historical template(s) selected for that patient. As another example, the computing device can select a template having metadata that corresponds to a patient medical history. In some implementations, a user may select a template such as based on template metadata and/or patient profile according to any of the examples discussed herein. Advantageously, selecting templates based on one or more criteria, such as demographics, may improve the accuracy of registering the template to the patient image and/or determining patient conditions. The selected template may represent an average or normal body portion for the demographics associated with the template. In some implementations, the selected template may comprise images of the patient, such as historical images. In some implementations, the computing device may select a single template to register to the patient image although other implementations are envisioned such as selecting fewer than ten templates, fewer than five templates, fewer than three templates, etc. Advantageously, the computing device may not need to select a large number of templates to register to the patient image in order to provide an accurate and robust system for analyzing patient medical images.

At block 205, the computing device can identify anatomical features of a template and/or patient image to be matched during registration. The anatomical features used to register the template can be pre-configured, dynamically configurable, and/or may be based on body region. In some implementations, a user may select one or more anatomical features to register the template, such as via an interactive graphical user interface. For example, a user may select ventricles, skull, and/or brain surface to match when registering a template to a patient image. The computing device can identify pixels associated with selected anatomical features. The computing device may not deform selected anatomical features during registration. For example, the computing device may not modify pixel values associated with selected anatomical features during registration and may modify pixel values not associated with selected anatomical features. In some implementations, the computing device may determine a degree to which a pixel, or region of pixels, is associated with a selected anatomical feature and may modify (or inhibit modifying) the pixel(s) based on the determined degree of association. Advantageously, registering templates to patient images based on pre-defined and/or user-selected anatomical features may improve a registration optimization.

At block 207, the computing device can register the template to the patient image. The computing device can register templates corresponding to various body parts. For example, the computing device may register a template corresponding to a skull, and a template corresponding to a brain surface, and a template corresponding to ventricles. The computing device can deformably and/or non-deformably register the template to the patient image. The computing device can register the template based on anatomical features. The computing device can match anatomical features in the template to anatomical features in the patient image. For example, when registering a template corresponding to a head, the computing device can match one or more anatomical features in the template, such as the skull, the brain surface, and/or ventricles, to corresponding features in the patient image. Registering a template to a patient image can comprise generating one or more registered images. A registered image can comprise a patient image and optionally a template or registered template. A registered image can comprise anatomical information associated with the template. Registering a template to a patient image can include assigning anatomical information from a template to a patient image.

Registering a template to a patient image can include modifying locations, sizes, shapes, etc. of anatomical features or regions of a template, which is generally referred to as transforming a template. As described herein, registration can include matching anatomical features in a template to corresponding anatomical features in a patient image. The computing device can match corresponding anatomical features by identifying pixels in the template that have similar or identical vales as pixels in the patient image in the same anatomical region. For example, the computing device may match ventricles in a brain by identifying pixels in a certain region of a template having similar or identical values to pixels in a corresponding region in a patient image. Registering a template can include rotating, tilting, translating, transforming, compressing, and/or stretching the template, or portions thereof, such as by modifying pixel values of the template, to best match a morphology of the patient image. In some implementations, non-deformable registration can include uniformly applying a transformation to a template. Applying a transformation uniformly to a template can include modifying pixel values to have the same values as other pixels in the template (e.g., shifting the pixel values). For example, non-deformable registration can include rotating and/or translating a template, or portions thereof. In some implementations, deformable registration can include non-uniformly applying a transformation, or multiple transformations, to a template. Applying a non-uniform transformation to a template can include modifying at least some pixel values independently of other pixel values in the template. For example, deformable registration can include compressing and/or stretching a template, or portions thereof. Registering a template can include aligning the template into a common coordinate frame as the patient image, such as based on anatomical features. In some implementations, the computing device can apply a transformation to a template comprising one or more image series. In some implementations, the computing device can apply different transformations to different image series of a template.

The computing device can implement one or more optimization processes to perform a registration process between a template and a patient image. In one implementation, an optimization process minimizes differences between a template and a patient image when registering the template to the patient image. A difference between a template and a patient image can be measured by a number of pixel values that are different between the registered template and the patient image and/or by a magnitude of differences between corresponding pixel values of the template and the patient image. An optimization process can minimize a global difference between a template and a patient image (e.g., a total difference for an entire template-patient image pair) or an optimization process can minimize local differences (e.g., differences at various portions within the templates/patient image pair), such as for preferred anatomical landmarks. For example, a local optimization process may preferentially registered certain anatomical features with a higher degree of matching than other anatomical features. With reference to an example head CT scan or x-ray, an example local optimization process could be configured to minimize a difference between certain anatomical structures, such as the skull (e.g., an outer boundary of the other anatomical features) and/or ventricles (e.g., a relatively central structure), between a head CT template and the head CT patient image. This local optimization process may result in a very close match between the anatomical landmarks (e.g., the skull and/or ventricles), but may result in a larger overall total difference than if global optimization was performed. However, such local optimization may advantageously expose anatomical regions where differences between the template and patient image are larger than expected and which may not have been discovered if each anatomical feature is considered (e.g., equally) in the registration process.

An example global optimization process can minimize a global difference between a template and a patient image for an entire anatomical region which may result in a close overall match between the entire set of anatomical landmarks (e.g., the skull and/or ventricles), but may result in larger differences for individual corresponding anatomical landmarks than if a local optimization was performed. However, global optimization may advantageously register a plurality of anatomical features within an anatomical region with an optimal degree of matching which may not have occurred if certain anatomical feature are preferentially registered in the registration process. Advantageously, registering a template to a patient image with process(es) that optimize for minimum global differences may provide close enough matching between anatomical features such as to identify anatomical features in the patient image from the template. Moreover, registering based on global optimization can advantageously improve patient diagnosis such as by providing a most accurate analysis of an entire anatomical region which may represent how various anatomical features within the anatomical region interact with and affect each other which can indicate health conditions.

In some implementations, the computing device may monitor and/or store operations and/or parameters associated with registering the template. For example, the computing device can store in memory transformations applied to a template during registration. The computing device may store a registered template that was generated from performing a registration. A registered template may have different pixel values than a template, such as pixel values that were modified from the template during registration. The computing device can determine and/or store differences between registered templates and templates. For example, the computing device can generate one or more data arrays and/or a multi-dimensional data array having data representing a difference in pixel data between a template and a registered template. Such an array of data may be referred to as a difference array and may be at least a portion of registration information. In some implementations, the computing device can determine and/or store differences between a template and a patient image in a similar manner. In some implementations, the computing device may determine and/or store differences in pixel data between a registered template and a patient image in a similar manner.

At block 209 the computing device can optionally register additional templates to the patient image, for example by performing any of the operations associated with blocks 203-207. The computing device may register a plurality of templates to determine which of the plurality of templates matches most closely to the patient image. The computing device can determine which template matches most closely to the patient image by analyzing differences between the template and a registered template generated from the registration, differences between the registered template and the patient image, and/or differences between the template and the patient image, such as with one or more difference arrays described at block 211. The computing device may register a plurality of templates to provide additional information for analyzing patient conditions.

At block 211, the computing device may determine one or more patient conditions based on registering one or more templates to the patient image. The computing device can determine a patient condition based on a numerical quantity associated with the registration. For example, the computing device can access registration information, such as a transformation applied to a template during registration and can determine a patient condition based on one or more numerical quantities associated with the transformation. Registration information can include a difference between a template and a registered template, a difference between a registered template and a patient image, and/or a difference between a template and a patient image. Differences between templates and/or patient images can be represented by data arrays. For example, a data array may comprise data indicating a difference between pixel values, such as between a template and a registered template. For example, if a template comprises a pixel with an RGB value of (110, 15, 200) and a registered template has a corresponding pixel with an RGB value of (100, 20, 220), then an array indicating a difference between the two may have a corresponding cell with a value of (10, 5, 200). An array indicating a difference may be referred to as a difference array. The computing device can analyze difference arrays to determine patient conditions. In some implementations, the computing device can determine patient conditions based on local differences. For example, the computing device can determine a number and/or percentage of pixel values that are different (and/or whose difference exceeds a threshold) within corresponding local regions between a template and a registered template. Determining local differences (e.g., by analyzing pixel values differences between templates and/or patient images) can indicate differences in anatomical features between a patient image and a template which may indicate patient conditions. For example, analyzing a local difference in pixel values between a patient image and a registered template and/or between a registered template and a template can indicate how an anatomical feature in a patient image differs in area and/or volume from a corresponding anatomical feature in a template which can indicate a condition of the anatomical feature (e.g., hypertrophy, atrophy, shifting, etc.) The computing device can determine deviations based on the registration. For example, the computing device can determine an amount or degree to which the patient's physiological anatomy deviates from an average or normal physiological anatomy represented by the template based on a numerical quantity of a transformation applied to the template to register the template to the patient image. As another example, the computing device can determine an amount or degree to which the patient's physiological anatomy deviates from a previous physiological anatomy of the same patient based on a difference between numerical quantities of transformations applied to the template to register the template to a previous patient image and to a current patient image.

The computing device can determine one or more characteristics of an anatomical feature. For example, anatomical features corresponding to a brain may include a skull, a brain surface, a lobe, a ventricle, a hemorrhage, a mass or abnormal growth such as a tumor, a midline, subdural lesion, epidural lesion, global and/or regional atrophy, global and/or regional hypertrophy, among others. Other anatomical features may correspond to other anatomical regions, such as heart, lungs, bones, muscles, digestive organs, lymphatic organs, circulatory organs, skin, and the like. Numerous anatomical features exist, which may not be specifically identified herein but which are contemplated as being within the scope of this disclosure. Example, anatomical feature characteristics can include size, location, displacement, or the like. Characteristics can include time-dependent characteristics such as movement, shifting, volume or size change, or the like.

In one example, the computing device can determine a size and/or location of a mass in the brain based on the registration. A mass can comprise an abnormality such as a lesion or tumor. The computing device can determine an effect the mass has on other anatomical features which may be referred to as a “mass effect”. For example, the mass may apply pressure to adjacent anatomical features. The computing device can determine a mass effect such as by determining a numerical quantity associated with applying a transformation to a template. For example, transforming a template during registration, such as deformable registration, may require stretching a portion of the template such that the template matches to a space occupied by the mass in the patient. Identifying one or more numerical quantities associated with the transformation, such as a direction and magnitude, can indicate an amount that the mass is displacing adjacent anatomical features. Accordingly, the computing device can determine a precise and quantifiable effect the mass is having on anatomical features. For example, the computing device may determine that the mass has displaced an adjacent anatomical feature by a certain distance. In some implementations, the computing device can monitor and/or determine a change in mass effect over time. For example, by registering a template to a patient image (or a historical patient image to a current patient image), the computing device can determine a difference in numerical quantities associated with applying transformations to perform the registrations. Advantageously, the computing device may precisely and objectively determine a health condition of a patient based on registration information. Advantageously, the computing device may implement the same analysis criteria to determine the patient condition from one analysis to another such that various patient analyses will not be subject to different analysis criteria.

As another example, the computing device can determine a distance by which a brain surface is displaced from a skull based on registration information. As another example, the computing device can determine a distance by which one or more ventricles are displaced from each other based on registration information. As another example, the computing device can determine a developmental delay in a child based on registration information. As another example, the computing device can detect or determine an amount of midline shift based on registration information. As another example, the computing device can whether the expected density on a CT (or pixel intensity on an MRI, or isotope uptake on a PET) in a region is abnormal or has changed over time based on registration information.

In some implementations, the computing device can determine one or more patient conditions based on the template selected to register to the patient image. For example, template metadata associated with a selected template may indicate one or more characteristics of the template, such as health conditions of a subject of the template. The computing device can access template metadata to determine patient conditions. As an example, the computing device may determine that a particular template resulted in the most optimal registration (e.g., fewest differences and/or closest match) to a patient image from among a plurality of templates that were registered. The computing device may therefore determine that the patient's health conditions will likely be more similar to health conditions indicated in the optimal template's metadata than health conditions indicated in the other templates' metadata. For example, if a template that optimizes registration to a patient image originates from a subject having a particular health condition, then the computing device may determine that the patient has that particular health condition. As another example, if a template that does not optimize registration to a patient image originates from a subject having a particular health condition, then the computing device may determine that the patient does not have that particular health condition.

In some implementations, the computing device may determine a patient condition based on registering a plurality of templates to patient image(s). For example, the computing device may register a series of templates originating from a same medical examination. Registering and/or analyzing a plurality of templates may improve medical diagnoses by analyzing other anatomical regions which may not have been the focus of the intended study. For example, analyzing a plurality of templates corresponding to a chest region may provide information relating a coronary artery in addition to information relating to pulmonary abnormalities. As another example, analyzing a plurality of templates of a head region may provide information relating to a dental abscess in addition to information relating to a brain.

At block 213 the computing device can display the patient image. The computing device may optionally display the patient image in combination with the template(s) used to register to the patient image. The computing device can display the patient image in combination with unregistered and/or registered template(s). For example, the computing device may display a template before it has been registered to the patient image and/or after it has been registered to the patient image (e.g., after a transformation has been applied to the template). In some implementations, the computing device can display the patient image in combination with a portion of a template. For example, the computing device may display the patient image in combination with less than all images of a template and/or with less than an entirety of one or more images of a template. The computing device can generate user interface data to display the patient image and/or template(s). The computing device can display the template(s) superimposed on the patient image. The computing device can display the template(s) adjacent to the patient image. The computing device may display the template(s) in combination with the patient image based on a user input, such as a user input via an interactive graphical user interface. For example, a user may view a patient image via a user interface and may select to toggle between displaying and not displaying a template. A user may select to view portions of a template and/or portions of an image associated with a template. A user may adjust other view preferences such as whether a template is displayed as overlaid on a patient image or adjacent to a patient image, a color or contrast of an image, a size of an image, or the like.

At block 215 the computing device can optionally display the patient image in combination with information relating to anatomical information of the template. As described previously herein, registering a template to a patient image can include assigning anatomical information to the patient image. The computing device can display anatomical information superimposed on and/or adjacent to a patient image. The computing device can display all anatomical information of a template or less than all of the anatomical information. The computing device can display anatomical information based on a user input, such as via an interactive graphical user interface. For example, a user may select to view or hide anatomical information (in whole or in part). As another example, the computing device may display anatomical information in response to a user selecting a corresponding portion of an image such as an anatomical feature. A user can select a portion of an image to display anatomical information such as by selecting the portion via a mouse and/or cursor, touching a touchscreen, hovering a cursor over the portion, voice commands, or the like. The computing device can display anatomical information with or without displaying a corresponding template. The computing device can display a template with or without displaying corresponding anatomical information.

At block 217, the computing device can optionally generate a report or a portion of a report. The computing device can generate a report based on accessing anatomical information. The computing device can generate a report based on a user selection of the patient image and/or template. For example, in response to a user selection of a portion of a patient image, the computing device can access anatomical information corresponding to the selected portion of the image. The report can include information from the anatomical label. The report can include registration information, such as one or more numerical quantities associated with a transformation. The report can include information relating to a condition of the patient, such as a condition detected or analyzed by the computing device based on registration information.

The computing device may determine a location in a report based on a user selection of a portion of the patient image. The computing device may automatically access a location of a report corresponding to a user selected portion of the patient image. In some implementations, the computing device may also automatically generate text for the identified location in the report. In some implementations, the computing device may display the determined location of the report to a user for the user to add text and/or review. In some implementations, the computing device may add text, such as from a user typing and/or dictating, to a location in a report corresponding to a user selected portion of the patient image. Accordingly, the computing device can store discreet data elements. Discreet data elements and/or data this is otherwise organized can improve the efficiency and accuracy of medical reporting, clinical research, tracking health condition progression or regression, or the like.

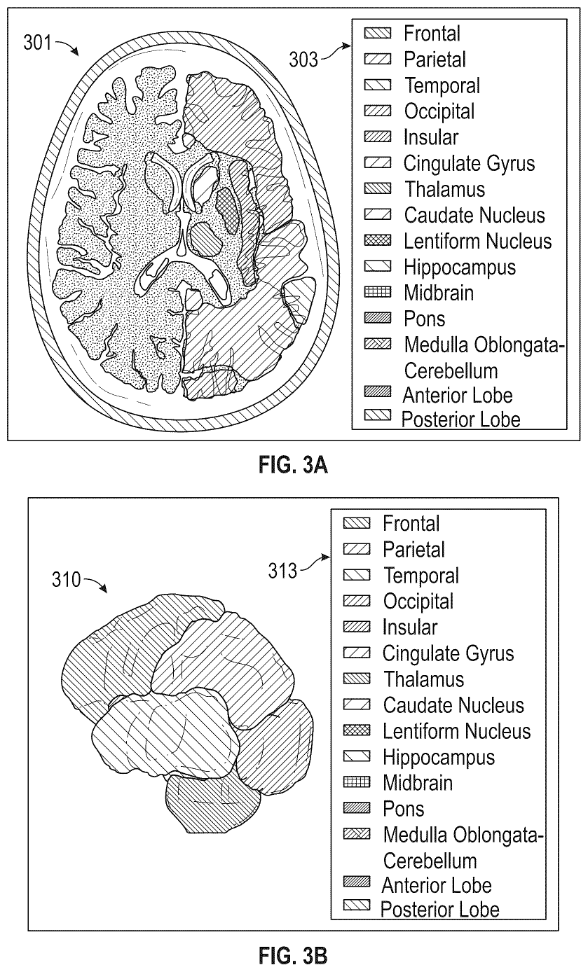

FIG. 3A illustrates an example image 301 of a template. The image 301 may be a 2D image. In this example, the image 301 corresponds to a brain. The image 301 may be part of an image series. For example, the image 301 may be one slice of plurality of images in a series. The image 301 comprises anatomical information 303. The anatomical information 303 can indicate various anatomical features represented in the image 301. The anatomical information 303 identify a name of anatomical features. In some implementations, the anatomical information 303 can identify other information associated with anatomical features such as size, location, health risks associated with the anatomical feature, biological composition of the anatomical feature, medication associated with the anatomical feature, or the like. A computing device can render the image 301 via a display to be viewed by a user. The user can select to display or hide one or more of the anatomical information 303. In some implementations, a user may add, remove, and/or modify one or more portions of the anatomical information 303 such as via a user interface.

FIG. 3B illustrates an example template 310. A computing device may generate the template 310 from one or more images, such as image 301. The template 310 may comprise a series of images. In this example, the template 310 is a 3D representation of a brain. The template 310 may comprise a series of images of one or more individuals. The template 310 may comprise a series of images generated using one or more imaging modalities. The template 310 may comprise anatomical information 313. A computing device may generate the anatomical information 313 from anatomical information associated with images of a series of images. In some implementations, a user may add, remove, and/or modify one or more portions of the anatomical information 313 such as via a user interface. Demographic information may be associated with the template 310. For example, the template 310 may represent average or normal characteristics of a brain for one or more given demographics.

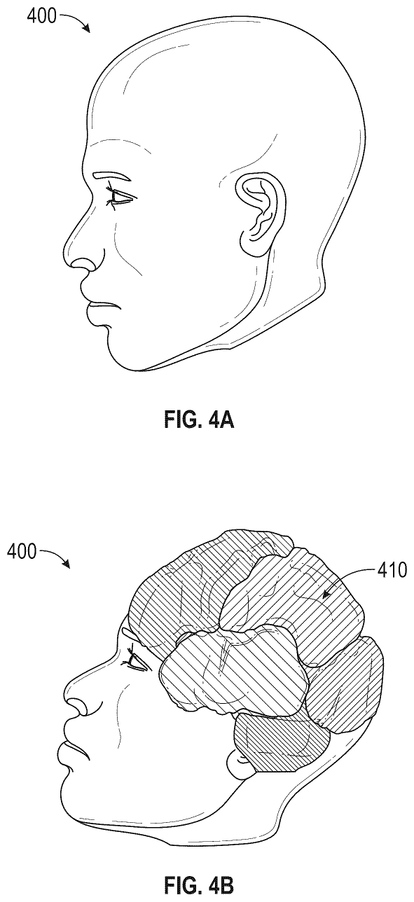

FIG. 4A illustrates an example patient representation 400. The patient representation 400 may be a 3D representation. In this example, the patient representation 400 corresponds to a head of a patient. A computing device may generate the patient representation 400 using one or more imaging modalities.

FIG. 4B illustrates an example template 410 that has been registered to the patient representation 400. A computing device may register the template 410 to the patient representation 400 using deformable and/or non-deformable registration. Registering the template 410 to the patient representation 400 can include matching a volume of the template 410 to a volume of the patient representation 400. Registering the template 410 to the patient representation 400 can include matching one or more anatomical features or landmarks of the template 410 to one or more corresponding anatomical features or landmarks of the patient representation 400. Registering the template 410 to the patient representation 400 can include registering (deformably and/or non-deformably) one or more images of a series of images of the template 410.

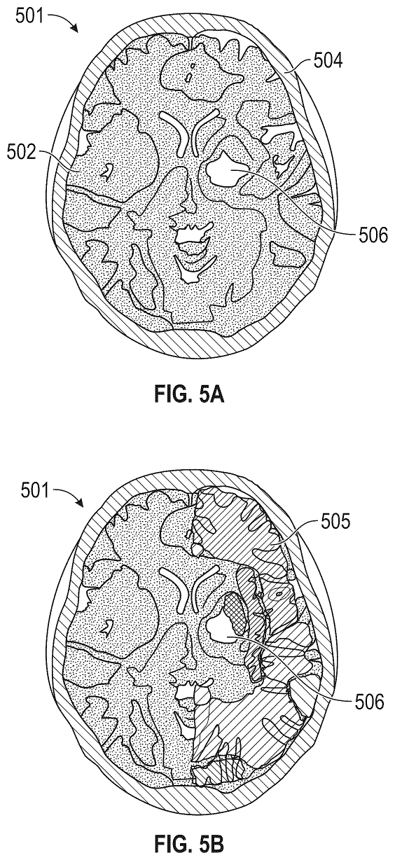

FIG. 5A illustrates an example patient image 501. In this example, the patient image 501 is a 2D representation of a head portion of a patient. The patient image 501 can show various anatomical features such as a brain 502, a skull 504, a hemorrhage 506, or other anatomical features. The patient image 501 may correspond to a 3D representation of the patient. The patient image 501 may be a single slice of a series of image slices of a 3D representation of the patient.

FIG. 5B illustrates the patient image 501 displayed in combination with a template 505. In this example, the template 505 is superimposed on the patient image 501. The template 505 may be a single image of a template or some other portion of an image. The template 505 may correspond to a registered template. For example, one or more transformations may have been applied to a template to generate a registered template to which the template 505 corresponds. A user may visualize the hemorrhage 506 and an effect it may be having on adjacent anatomical features indicated by anatomical information. A computing device may determine a quantifiable effect the hemorrhage 506 is having on adjacent tissues based on registering a template to the patient image 501. In some implementations, the computing device may cause display of anatomical information, such as shown and/or described at FIG. 3A. The computing device can display the anatomical information in response to a user input, such as user hovering a cursor of a regions of the patient image 501. In some implementations, the computing device can display one or more portions of the template 505 in response to a user input, such as user hovering a cursor over regions of the patient image 501.

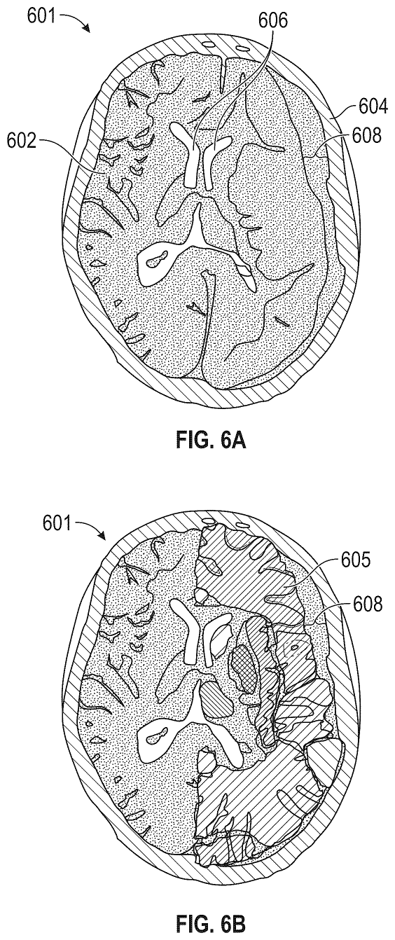

FIG. 6A illustrates an example patient image 601. In this example, the patient image 601 is a 2D representation of a head portion of a patient. The patient image 601 can show various anatomical features such as a brain 602, a skull 604, ventricles 606, and/or other anatomical features. The patient image 601 may be a slice of a series of image slices that collectively create a 3D imaging volume or the image 601 may be a 2D construction of a particular plane of a 3D imaging volume. In this example, the patient has a midline shift represented by space 608 between the brain 602 and the skull 604. shown in the patient image 601. The midline shift may be caused by intracranial pressure such as from a right chronic hematoma causing shift of the brain to the left.

FIG. 6B illustrates the patient image 601 displayed in combination with a template 605. In this example, the template 605 is superimposed on the patient image 601. The template 605 shown in FIG. 6B includes only a right side of the head (e.g., a “hemi-template”), but in other embodiment a template may correspond to more, or less, of the anatomical features of a medical image. The template 605 may correspond to a registered template. For example, one or more transformations may have been applied to a template to generate a registered template to which the template 605 corresponds. The template 605 may be registered to the patient image 601 based on matching corresponding anatomical features. For example, a computing device may register the template 605 based on matching ventricles, a brain surface, and/or the skull, to corresponding features in the patient image 601. A computing device may determine one or more conditions of the patient based on registering the template 605 to the patient image 601. For example, a computing device may detect and/or quantify a midline shift in the patient based on identifying one or more numerical quantities associated with applying one or more transformations to the template 605 to register the template 605 to the patient image 601. In some implementations, the computing device may cause display of anatomical information, such as shown and/or described at FIG. 3A. The computing device can display the anatomical information in response to a user input, such as user hovering a cursor of a regions of the patient image 601. In some implementations, the computing device can display one or more portions of the template 605 in response to a user input, such as user hovering a cursor over regions of the patient image 601.

### Example Implementations

Examples of the implementations of the present disclosure can be described in view of the following example clauses. The features recited in the below example implementations can be combined with additional features disclosed herein. Furthermore, additional inventive combinations of features are disclosed herein, which are not specifically recited in the below example implementations, and which do not include the same features as the specific implementations below. For sake of brevity, the below example implementations do not identify every inventive aspect of this disclosure. The below example implementations are not intended to identify key features or essential features of any subject matter described herein. Any of the example clauses below, or any features of the example clauses, can be combined with any one or more other example clauses, or features of the example clauses or other features of the present disclosure.

Clause 1. A system for facilitating medical image analysis, the system comprising: one or more hardware processors configured to: select, based on at least a patient profile of a patient, a template comprising indications of a plurality of anatomical regions of patient anatomy; register the template to a patient medical image of the patient based on at least matching a first of the anatomical regions in the template to a corresponding first anatomical region in the patient medical image; generate registration information based on registering the template to the patient medical image, the registration information comprising a plurality of numerical indicators of differences between corresponding anatomical regions in the registered template and the patient medical image; and determine a patient condition based on one or more of the numerical indicators.

Clause 2. The system of clause 1, wherein the first anatomical region is a landmark anatomical region.

Clause 3. The system of clause 2, wherein first anatomical feature is a landmark anatomical feature.

Clause 4. The system of clause 1, wherein said registering the template to the patient medical image comprises one or more of resizing or rotating the template.

Clause 5. The system of clause 1, wherein said registering the template to the patient medical image comprises one or more of deformable or non-deformable registration.

Clause 6. The system of clause 1, wherein numerical indicators indicate a difference in size, area, or volume.

Clause 7. The system of clause 1, wherein said registering the template to the patient medical image comprises generating a registered template by modifying pixel values of the template.

Clause 8. The system of clause 1, wherein said registering the template to the patient medical image comprises implementing an optimization process to minimize a difference between pixel values of the first anatomical region and the template.

Clause 9. The system of clause 1, wherein the numerical indicators indicate differences between pixel values of the template and the registered template.

Clause 10. The system of clause 9, wherein the numerical indicators include one or more of a quantity or percentage of the pixel values of the registered template that differ, within a threshold, from the pixel values of the template.

Clause 11. The system of clause 10, wherein one or more of the quantity or percentage of the pixel values that differ indicates an area or volume of an anatomical feature.

Clause 12. The system of clause 1, wherein the one or more hardware processors are further configured to: generate second registration information based on registering a second template to the patient medical image, wherein first metadata associated with the template indicates a first patient condition and second metadata associated with the second template indicates a second patient condition; determine an optimal template based on analyzing the registration information and the second registration information, the optimal template corresponding to the template or the second template; and determine the patient condition based on at least template metadata associated with the optimal template.

Clause 13. The system of clause 1 wherein the patient condition comprises one or more of a structural abnormality, an effect of an anatomical feature on adjacent anatomical features, a size and/or location of an anatomical feature, a change in a size of an anatomical feature, or a location of an anatomical feature.

Clause 14. The system of clause 1 wherein the template comprises anatomical information.

Clause 15. The system of clause 14 wherein registering the template to the patient medical image comprises associating the anatomical information to one or more anatomical features of the patient medical image.

Clause 16. The system of clause 14 wherein the one or more hardware processors are further configured to cause a display to render a user interface comprising the patient medical image and at least some of the anatomical information, the anatomical information displayed with reference to anatomical features of the patient medical image.

Clause 17. The system of clause 1 wherein the one or more hardware processors are further configured to cause a display to render a user interface comprising the patient medical image and at least a portion of the template superimposed on the patient medical image.

Clause 18. The system of clause 14 wherein the one or more hardware processors are further configured to identify a location in a report corresponding to an anatomical label, the anatomical label corresponding to an anatomical feature of the patient medical image selected by a user.

Clause 19. The system of clause 18 wherein the one or more hardware processors are further configured to cause a display to render a user interface comprising at least the identified location in the report.

Clause 20. The system of clause 18 wherein the one or more hardware processors are further configured to generate a textual description of the anatomical feature, the textual description based on at least the anatomical label.

Clause 21. The system of clause 20 wherein the one or more hardware processors are further configured to correlate the textual description of the anatomical feature to the identified location in the report.

Clause 22. The system of clause 1 wherein the patient profile comprises one or more of patient demographics, patient medical history, or a template that was previously associated with the patient.

Clause 23. A computerized method, performed by a computing system having one or more hardware computer processors and one or more non-transitory computer readable storage device storing software instructions executable by the computing system to perform the computerized method comprising: accessing a medical image of a patient; determining, based on one or more characteristics of a patient profile, at least a first template associated with a first patient condition and a second template associated with a second patient condition; transforming the first template into a first registered template by registering an anatomical landmark in each of the first template and the medical image; determining a first quantifier indicating a first difference between the first registered template and the medical image; transforming the second template into a second registered template by registering the anatomical landmark in each of the second template and the medical image; determining a second quantifier indicating a second difference between the second registered template and the medical image; and if the first quantifier is less than the second quantifier, indicating that the first patient condition is more likely associated with the patient than the second patient condition; or if the second quantifier is less than the first quantifier, indicating that the second patient condition is more likely associated with the patient than the first patient condition.

Clause 24. The computerized method of clause 23, wherein the patient profile indicates demographic information, including one or more of age, weight, height, gender, or ethnicity, and medical information, including one or more of medical history, past or present diseases, physiological condition, past medical treatments, surgical procedures undergone, lab results, or medications prescribed and taken.

### Additional Implementations

As used herein, “real-time” or “substantial real-time” may refer to events (e.g., receiving, processing, transmitting, displaying etc.) that occur at the same time or substantially the same time (e.g., neglecting any small delays such as those that are imperceptible and/or inconsequential to humans such as delays arising from electrical conduction or transmission). As a non-limiting example, “real-time” may refer to events that occur within a time frame of each other that is on the order of milliseconds, seconds, tens of seconds, or minutes. For example, “real-time” may refer to events that occur within a time frame of less than 1 minute, less than 30 seconds, less than 10 seconds, less than 1 second, less than 0.05 seconds, less than 0.01 seconds, less than 0.005 seconds, less than 0.001 seconds, etc. In some implementations, “real-time” may refer to events that occur at a same time as, or during, another event.

As used herein, “system,” “instrument,” “apparatus,” and “device” generally encompass both the hardware (for example, mechanical and electronic) and, in some implementations, associated software (for example, specialized computer programs for graphics control) components.

It is to be understood that not necessarily all objects or advantages may be achieved in accordance with any particular embodiment described herein. Thus, for example, those skilled in the art will recognize that certain implementations may be configured to operate in a manner that achieves or optimizes one advantage or group of advantages as taught herein without necessarily achieving other objects or advantages as may be taught or suggested herein.

Each of the processes, methods, and algorithms described in the preceding sections may be embodied in, and fully or partially automated by, code modules executed by one or more computer systems or computer processors including computer hardware. The code modules may be stored on any type of non-transitory computer-readable medium or computer storage device, such as hard drives, solid state memory, optical disc, and/or the like. The systems and modules may also be transmitted as gencrated data signals (for example, as part of a carrier wave or other analog or digital propagated signal) on a variety of computer-readable transmission mediums, including wireless-based and wired/cable-based mediums, and may take a variety of forms (for example, as part of a single or multiplexed analog signal, or as multiple discrete digital packets or frames). The processes and algorithms may be implemented partially or wholly in application-specific circuitry. The results of the disclosed processes and process steps may be stored, persistently or otherwise, in any type of non-transitory computer storage such as, for example, volatile or non-volatile storage.

Many other variations than those described herein will be apparent from this disclosure. For example, depending on the embodiment, certain acts, events, or functions of any of the algorithms described herein can be performed in a different sequence, can be added, merged, or left out altogether (for example, not all described acts or events are necessary for the practice of the algorithms). Moreover, in certain implementations, acts or events can be performed concurrently, for example, through multi-threaded processing, interrupt processing, or multiple processors or processor cores or on other parallel architectures, rather than sequentially. In addition, different tasks or processes can be performed by different machines and/or computing systems that can function together.

The various illustrative logical blocks, modules, and algorithm elements described in connection with the implementations disclosed herein can be implemented as electronic hardware, computer software, or combinations of both. To clearly illustrate this interchangeability of hardware and software, various illustrative components, blocks, modules, and elements have been described herein generally in terms of their functionality. Whether such functionality is implemented as hardware or software depends upon the particular application and design constraints imposed on the overall system. The described functionality can be implemented in varying ways for each particular application, but such implementation decisions should not be interpreted as causing a departure from the scope of the disclosure.

The various features and processes described herein may be used independently of one another, or may be combined in various ways. All possible combinations and sub-combinations are intended to fall within the scope of this disclosure. In addition, certain method or process blocks may be omitted in some implementations. The methods and processes described herein are also not limited to any particular sequence, and the blocks or states relating thereto can be performed in other sequences that are appropriate. For example, described blocks or states may be performed in an order other than that specifically disclosed, or multiple blocks or states may be combined in a single block or state. The example blocks or states may be performed in serial, in parallel, or in some other manner. Blocks or states may be added to or removed from the disclosed example implementations. The example systems and components described herein may be configured differently than described. For example, elements may be added to, removed from, or rearranged compared to the disclosed example implementations.

The various illustrative logical blocks and modules described in connection with the implementations disclosed herein can be implemented or performed by a machine, such as a general purpose processor, a digital signal processor (“DSP”), an application specific integrated circuit (“ASIC”), a field programmable gate array (“FPGA”) or other programmable logic device, discrete gate or transistor logic, discrete hardware components, or any combination thereof designed to perform the functions described herein. A general purpose processor can be a microprocessor, but in the alternative, the processor can be a controller, microcontroller, or state machine, combinations of the same, or the like. A processor can include electrical circuitry configured to process computer-executable instructions. In another embodiment, a processor includes an FPGA or other programmable devices that performs logic operations without processing computer-executable instructions. A processor can also be implemented as a combination of computing devices, for example, a combination of a DSP and a microprocessor, a plurality of microprocessors, one or more microprocessors in conjunction with a DSP core, or any other such configuration. Although described herein primarily with respect to digital technology, a processor may also include primarily analog components. For example, some, or all, of the signal processing algorithms described herein may be implemented in analog circuitry or mixed analog and digital circuitry. A computing environment can include any type of computer system, including, but not limited to, a computer system based on a microprocessor, a mainframe computer, a digital signal processor, a portable computing device, a device controller, or a computational engine within an appliance, to name a few.

The elements of a method, process, or algorithm described in connection with the implementations disclosed herein can be embodied directly in hardware, in a software module stored in one or more memory devices and executed by one or more processors, or in a combination of the two. A software module can reside in RAM memory, flash memory, ROM memory, EPROM memory, EEPROM memory, registers, hard disk, a removable disk, a CD-ROM, or any other form of non-transitory computer-readable storage medium, media, or physical computer storage known in the art. An example storage medium can be coupled to the processor such that the processor can read information from, and write information to, the storage medium. In the alternative, the storage medium can be integral to the processor. The storage medium can be volatile or nonvolatile. The processor and the storage medium can reside in an ASIC. The ASIC can reside in a user terminal. In the alternative, the processor and the storage medium can reside as discrete components in a user terminal.

Conditional language, such as, among others, “can,” “could,” “might,” or “may,” unless specifically stated otherwise, or otherwise understood within the context as used, is generally intended to convey that certain implementations include, while other implementations do not include, certain features, elements and/or steps. Thus, such conditional language is not generally intended to imply that features, elements and/or steps are in any way required for one or more implementations or that one or more implementations necessarily include logic for deciding, with or without user input or prompting, whether these features, elements and/or steps are included or are to be performed in any particular embodiment.

Disjunctive language such as the phrase “at least one of X, Y, or Z,” unless specifically stated otherwise, is otherwise understood with the context as used in general to present that an item, term, and so forth, may be either X, Y, or Z, or any combination thereof (for example, X, Y, and/or Z). Thus, such disjunctive language is not generally intended to, and should not, imply that certain implementations require at least one of X, at least one of Y, or at least one of Z to each be present.

Language of degree used herein, such as the terms “approximately,” “about,” “generally,” and “substantially” as used herein represent a value, amount, or characteristic close to the stated value, amount, or characteristic that still performs a desired function or achieves a desired result. For example, the terms “approximately”, “about”, “generally,” and “substantially” may refer to an amount that is within less than 10% of, within less than 5% of, within less than 1% of, within less than 0.1% of, and within less than 0.01% of the stated amount. As another example, in certain implementations, the terms “generally parallel” and “substantially parallel” refer to a value, amount, or characteristic that departs from exactly parallel by less than or equal to 10 degrees, 5 degrees, 3 degrees, or 1 degree. As another example, in certain implementations, the terms “generally perpendicular” and “substantially perpendicular” refer to a value, amount, or characteristic that departs from exactly perpendicular by less than or equal to 10 degrees, 5 degrees, 3 degrees, or 1 degree.

Any process descriptions, elements, or blocks in the flow diagrams described herein and/or depicted in the attached figures should be understood as potentially representing modules, segments, or portions of code which include one or more executable instructions for implementing specific logical functions or steps in the process. Alternate implementations are included within the scope of the implementations described herein in which elements or functions may be deleted, executed out of order from that shown or discussed, including substantially concurrently or in reverse order, depending on the functionality involved, as would be understood by those skilled in the art.

Unless otherwise explicitly stated, articles such as “a” or “an” should generally be interpreted to include one or more described items. Accordingly, phrases such as “a device configured to” are intended to include one or more recited devices. Such one or more recited devices can also be collectively configured to carry out the stated recitations. For example, “a processor configured to carry out recitations A, B and C” can include a first processor configured to carry out recitation A working in conjunction with a second processor configured to carry out recitations B and C.

All of the methods and processes described herein may be embodied in, and partially or fully automated via, software code modules executed by one or more general purpose computers. For example, the methods described herein may be performed by the computing system and/or any other suitable computing device. The methods may be executed on the computing devices in response to execution of software instructions or other executable code read from a tangible computer readable medium. A tangible computer readable medium is a data storage device that can store data that is readable by a computer system. Examples of computer readable mediums include read-only memory, random-access memory, other volatile or non-volatile memory devices, CD-ROMs, magnetic tape, flash drives, and optical data storage devices.

It should be emphasized that many variations and modifications may be made to the herein-described implementations, the elements of which are to be understood as being among other acceptable examples. All such modifications and variations are intended to be included herein within the scope of this disclosure. The section headings used herein are merely provided to enhance readability and are not intended to limit the scope of the implementations disclosed in a particular section to the features or elements disclosed in that section. The foregoing description details certain implementations. It will be appreciated, however, that no matter how detailed the foregoing appears in text, the systems and methods can be practiced in many ways. As is also stated herein, it should be noted that the use of particular terminology when describing certain features or aspects of the systems and methods should not be taken to imply that the terminology is being re-defined herein to be restricted to including any specific characteristics of the features or aspects of the systems and methods with which that terminology is associated.

Those of skill in the art would understand that information, messages, and signals may be represented using any of a variety of different technologies and techniques. For example, data, instructions, commands, information, signals, bits, symbols, and chips that may be referenced throughout the above description may be represented by voltages, currents, electromagnetic waves, magnetic fields or particles, optical fields or particles, or any combination thereof.

What is claimed is:

1. A system for facilitating medical image analysis, the system comprising:
one or more hardware processors configured to:
select, based on at least a patient profile of a patient, a template comprising indications of a plurality of anatomical regions of patient anatomy;
register the template to a patient medical image of the patient based on at least matching a first of the anatomical regions in the template to a corresponding first anatomical region in the patient medical image;
generate registration information based on registering the template to the patient medical image, the registration information comprising a plurality of numerical indicators of differences between corresponding anatomical regions in the registered template and the patient medical image; and
determine a patient condition based on one or more of the numerical indicators.

2. The system of claim 1, wherein the first anatomical region is a landmark anatomical region.

3. The system of claim 2, wherein first anatomical feature is a landmark anatomical feature.

4. The system of claim 1, wherein said registering the template to the patient medical image comprises one or more of resizing or rotating the template.

5. The system of claim 1, wherein said registering the template to the patient medical image comprises one or more of deformable or non-deformable registration.

6. The system of claim 1, wherein numerical indicators indicate a difference in size, area, or volume.

7. The system of claim 1, wherein said registering the template to the patient medical image comprises generating a registered template by modifying pixel values of the template.

8. The system of claim 1, wherein said registering the template to the patient medical image comprises implementing an optimization process to minimize a difference between pixel values of the first anatomical region and the template.

9. The system of claim 1, wherein the numerical indicators indicate differences between pixel values of the template and the registered template.

10. The system of claim 9, wherein the numerical indicators include one or more of a quantity or percentage of the pixel values of the registered template that differ, within a threshold, from the pixel values of the template.

11. The system of claim 10, wherein one or more of the quantity or percentage of the pixel values that differ indicates an area or volume of an anatomical feature.

12. The system of claim 1, wherein the one or more hardware processors are further configured to:
generate second registration information based on registering a second template to the patient medical image, wherein first metadata associated with the template indicates a first patient condition and second metadata associated with the second template indicates a second patient condition;
determine an optimal template based on analyzing the registration information and the second registration information, the optimal template corresponding to the template or the second template; and
determine the patient condition based on at least template metadata associated with the optimal template.

13. The system of claim 1 wherein the patient condition comprises one or more of a structural abnormality, an effect of an anatomical feature on adjacent anatomical features, a size and/or location of an anatomical feature, a change in a size of an anatomical feature, or a location of an anatomical feature.

14. The system of claim 1 wherein the template comprises anatomical information.

15. The system of claim 14 wherein registering the template to the patient medical image comprises associating the anatomical information to one or more anatomical features of the patient medical image.

16. The system of claim 14 wherein the one or more hardware processors are further configured to cause a display to render a user interface comprising the patient medical image and at least some of the anatomical information, the anatomical information displayed with reference to anatomical features of the patient medical image.

17. The system of claim 1 wherein the one or more hardware processors are further configured to cause a display to render a user interface comprising the patient medical image and at least a portion of the template superimposed on the patient medical image.

18. The system of claim 14 wherein the one or more hardware processors are further configured to identify a location in a report corresponding to an anatomical label, the anatomical label corresponding to an anatomical feature of the patient medical image selected by a user.

19. The system of claim 18 wherein the one or more hardware processors are further configured to cause a display to render a user interface comprising at least the identified location in the report.

20. The system of claim 18 wherein the one or more hardware processors are further configured to generate a textual description of the anatomical feature, the textual description based on at least the anatomical label.

21. The system of claim 20 wherein the one or more hardware processors are further configured to correlate the textual description of the anatomical feature to the identified location in the report.

22. The system of claim 1 wherein the patient profile comprises one or more of patient demographics, patient medical history, or a template that was previously associated with the patient.

23. A computerized method, performed by a computing system having one or more hardware computer processors and one or more non-transitory computer readable storage device storing software instructions executable by the computing system to perform the computerized method comprising:
accessing a medical image of a patient;
determining, based on one or more characteristics of a patient profile, at least a first template associated with a first patient condition and a second template associated with a second patient condition;
transforming the first template into a first registered template by registering an anatomical landmark in each of the first template and the medical image;
determining a first quantifier indicating a first difference between the first registered template and the medical image;
transforming the second template into a second registered template by registering the anatomical landmark in cach of the second template and the medical image;
determining a second quantifier indicating a second difference between the second registered template and the medical image; and
if the first quantifier is less than the second quantifier, indicating that the first patient condition is more likely associated with the patient than the second patient condition; or if the second quantifier is less than the first quantifier, indicating that the second patient condition is more likely associated with the patient than the first patient condition.

24. The computerized method of claim 23, wherein the patient profile indicates demographic information, including one or more of age, weight, height, gender, or ethnicity, and medical information, including one or more of medical history, past or present diseases, physiological condition, past medical treatments, surgical procedures undergone, lab results, or medications prescribed and taken.
